(19) United States
(12) Patent Application Publication
(10) Pub. No.: US 2026/0074054 A1
(43) Pub. Date: Mar. 12, 2026

(54) METHOD, APPARATUS, AND COMPUTER PROGRAM FOR ALIGNING AUGMENTED REALITY OBJECT BASED ON VOXEL MARKERS

(71) Applicant: DECASIGHT CORPORATION, Seoul (KR)

(72) Inventors: Sooho CHOI, Ansan-si (KR); Kiwook LEE, Incheon (KR); Seungri PARK, Seoul (KR)

(21) Appl. No.: 19/086,669

(22) Filed: Mar. 21, 2025

(30) Foreign Application Priority Data
Sep. 9, 2024 (KR) 10-2024-0122273

(51) Int. Cl. G16H 30/40; G06T 3/14; G06T 7/33; G06V 10/46

(52) U.S. Cl. CPC G16H 30/40; G06T 3/14; G06T 7/344; G06V 10/46; G06T 2207/10081; G06T 2207/30204

(57) ABSTRACT

The present disclosure relates to a method, apparatus, and computer program for registering an augmented reality object, based on a voxel marker. The method of registering an augmented reality object, based on a voxel marker includes performing a CT scan of a target object to which the voxel marker is attached, performing segmentation by which only a region of interest of the target object and the voxel marker are extracted from a CT scan image, reconstructing the segmented CT scan image into a 3D model, and performing registration by aligning a voxel marker generated by the CT scan to the location, size, and rotation of a template voxel marker.

### CROSS-REFERENCE TO RELATED APPLICATION

This application claims priority under 35 U.S.C. § 119 to Korean Patent Application No. 10-2024-0122273 filed in the Korean Intellectual Property Office on Sep. 9, 2024, the entire contents of which are hereby incorporated by reference.

### Technical Field

The present disclosure relates to a method, apparatus, and computer program for registering an augmented reality object, based on a voxel marker and, more specifically, to a method, apparatus, and computer program for registering an augmented reality object by using a 3D voxel marker including multiple materials having different densities.

### Background Art

Surgery is a medical procedure that involves making incisions in the body to treat diseases or injuries, and thus greatly relies on the experience of a surgeon. Recently, surgical navigation linked with augmented reality technology functions by visually guiding surgical instruments to avoid critical anatomical structures and accurately target the affected site, thereby enhancing the safety and precision of the surgery.

To effectively utilize such surgical navigation, a registration process is required, in which anatomical structure information obtained from a CT scan is converted into a 3D model and the 3D model is precisely registered with an actual object, which is a patient.

A point-based registration method using the existing IR markers may cause inaccuracy in registration due to errors in the central coordinates of the IR marker scan model, which arise from the CT slice interval and imaging direction. This inaccuracy in registration means that errors occur in the patient's anatomical structure information during surgery, which increases the risks of surgery.

Even when registration is performed using ArUco markers instead of IR markers, the existing ArUco markers are 2D printed on the surfaces of the markers. Therefore, the markers may not be detected in a CT scan and the patterns thereof may be invisible.

In conclusion, despite the difficulty in precise registration between a scan model and an actual model due to the limitations of the existing IR markers and ArUco markers, no appropriate solution or proposal has been presented yet.

### DISCLOSURE

### Technical Problem

The present disclosure is made to solve the problems of the above-mentioned related art, and is to provide a method, apparatus, and computer program for registering an augmented reality object, based on a voxel marker.

In addition, the present disclosure is to provide a method, apparatus, and computer program for registering an augmented reality object by using a three-dimensional voxel marker including multiple materials having different densities.

In addition, the present disclosure is to provide a method, apparatus, and computer program for registering an augmented reality object, based on a voxel marker capable of providing precise registration between a scan model and an actual model.

The technical problems to be solved in the present disclosure are not limited to the technical problems mentioned above, and other technical problems that are not mentioned may be clearly understood by those of ordinary skill in the art to which the present disclosure belongs from the contents described in this specification.

### Technical Solution

According to a first aspect of the present disclosure, a method of registering an augmented reality object, based on a voxel marker may include performing a CT scan of a target object to which a voxel marker is attached, performing segmentation by which only a region of interest of the target object and the voxel marker are extracted from a CT scan image, reconstructing the segmented CT scan image into a 3D model, and performing registration by aligning a voxel marker generated by the CT scan to the location, size, and rotation of a template voxel marker.

The voxel marker may include multiple types of voxels having different densities.

The region of interest may include an affected area of a patient or a surgical target area.

The template voxel marker may be an ideal 3D model voxel marker that reflects the location, size, and rotation of the voxel marker.

In addition, the performing of the registration may include performing primary registration and secondary registration. The performing of the primary registration may include registering the voxel marker generated by a CT scan to register the same to the template voxel marker by applying a rigid body transformation matrix thereto, and the performing of the secondary registration may include performing registration to minimize a brightness difference between each voxel included in the voxel marker generated by a CT scan and each voxel included in the template voxel marker.

A second aspect of the present disclosure may provide a computer program stored in a medium for executing, in combination with hardware, a method of registering an augmented reality object, based on a voxel marker.

A third aspect of the present disclosure may provide an apparatus for registering an augmented reality object, based on a voxel marker, the apparatus including a processor, wherein the processor may be configured to execute a CT scan of a target object to which a voxel marker is attached, segmentation by which only a region of interest of the target object and the voxel marker are extracted from a CT scan image, reconstruction of the segmented CT scan image into a 3D model, and registration by aligning the voxel marker generated by the CT scan with the location, size, and rotation of a template voxel marker.

The voxel marker may include multiple types of voxels having different densities.

The region of interest may include an affected area of a patient or a surgical target area.

The template voxel marker may be an ideal 3D model voxel marker that reflects the location, size, and rotation of the voxel marker.

In addition, the executing of the registration may include executing primary registration and secondary registration. The executing of the primary registration may include registering the voxel marker generated by a CT scan to the template voxel marker by applying a rigid body transformation matrix thereto, and the executing of the secondary registration may include executing registration to minimize a brightness difference between each voxel included in the voxel marker generated by a CT scan and each voxel included in the template voxel marker.

### Advantageous Effects

Accordingly, a method of registering an augmented reality object by using a 3D voxel marker including multiple materials having different densities is provided in the method, apparatus, and computer program for registering an augmented reality object, based on a voxel marker according to an embodiment of the present disclosure.

In addition, a method of registering an augmented reality object, based on a voxel marker capable of providing precise registration between a scan model and an actual model is provided in the method, apparatus, and computer program for registering an augmented reality object, based on a voxel marker according to an embodiment of the present disclosure.

The effects obtainable in the present disclosure are not limited to the effects mentioned above, and other effects not mentioned will be clearly understood by those skilled in the art to which the present disclosure belongs from the contents described in this specification.

### DESCRIPTION OF DRAWINGS

The accompanying drawings, which are included as part of the detailed description to help understanding of the present disclosure, provide examples of the present disclosure and explain the technical idea of the present disclosure together with the detailed description.

FIG. 1 illustrates an infrared (IR) marker and an infrared camera which are used in IR marker-based augmented reality object registration of the related art.

FIG. 2 illustrates an ArUco marker used in ArUco marker-based augmented reality object registration of the related art.

FIG. 3 is a flowchart illustrating a method of registering an augmented reality object, based on a voxel marker according to an embodiment of the present disclosure.

FIG. 4 illustrates a template voxel marker manufactured according to an embodiment of the present disclosure.

FIG. 5 illustrates a 3D printing filament material used in manufacturing a voxel-based marker having a density difference obtained through a combination of materials according to an embodiment of the present disclosure.

FIG. 6 illustrates a 2D scan image obtained by CT scanning a target object to which a voxel-based 3D marker is attached, according to an embodiment of the present disclosure.

FIG. 7 illustrates an image that has undergone segmentation by which only a region of interest of a target object and a voxel marker are extracted from a CT scan image according to an embodiment of the present disclosure.

FIG. 8 illustrates a 3D model generated by integrating multiple 2D CT scan images that have undergone segmentation according to an embodiment of the present disclosure.

FIG. 9 illustrates a flowchart for aligning and registering a voxel marker generated by a CT scan with a template voxel marker according to an embodiment of the present disclosure.

FIG. 10 is a flowchart illustrating operations constituting primary registration and secondary registration by which a voxel marker generated by a CT scan is aligned and registered with a template voxel marker according with an embodiment of the present disclosure.

FIG. 11 illustrates a primary registration process of aligning a voxel marker generated by a CT scan with a template voxel marker according to an embodiment of the present disclosure.

FIG. 12 illustrates a primary registration result obtained by aligning a voxel marker generated by a CT scan with a template voxel marker according to an embodiment of the present disclosure.

FIG. 13 illustrates a secondary registration result obtained by aligning a voxel marker generated by CT scans with a template voxel marker according to an embodiment of the present disclosure.

FIG. 14 illustrates an apparatus to which a proposed method of the present disclosure is applicable.

### MODE FOR INVENTION

Hereinafter, the embodiments disclosed in this specification will be described in detail with reference to the attached drawings. The purpose, specific advantages, and novel features of the present disclosure will become more apparent from the following detailed description and preferred embodiments associated with the attached drawings.

In advance, the terms or words used in this specification and claims are appropriately defined by the inventor to explain his/her own invention in the best possible way, and should be interpreted as meanings and concepts that are consistent with the technical idea of the present disclosure, and are only for explaining the embodiments, and should not be construed as limiting the present disclosure.

When assigning reference signs to components, the same or similar components will be assigned the same reference signs regardless of the reference signs, and redundant descriptions thereof will be omitted. The suffixes “module” and “part” used for components in the following description are assigned or used interchangeably in consideration of the case of writing the specification, and do not have distinct meanings or roles in themselves, and may mean software or hardware components.

In describing components of the present disclosure, if a component is expressed in singular form, it should be understood to include plural forms unless specifically stated otherwise. In addition, terms such as “first,” “second,” etc. are used to distinguish one component from another component, and the components are not limited by the terms. In addition, when a component is connected to another component, it means that another component may be connected between the component and the other component.

In addition, in describing the embodiments disclosed in this specification, if it is determined that a specific description of a related known technology may obscure the gist of the embodiments disclosed in this specification, the detailed description thereof will be omitted. In addition, the attached drawings are only intended to facilitate easy understanding of the embodiments disclosed in this specification, and the technical ideas disclosed in this specification are not limited by the attached drawings, and it should be understood to include all modifications, equivalents, and substitutes included in the spirit and technical scope of the present disclosure.

Hereinafter, exemplary embodiments of a method, apparatus, and computer program for registering an augmented reality object, based on a voxel marker according to the present disclosure are described in detail with reference to the attached drawings.

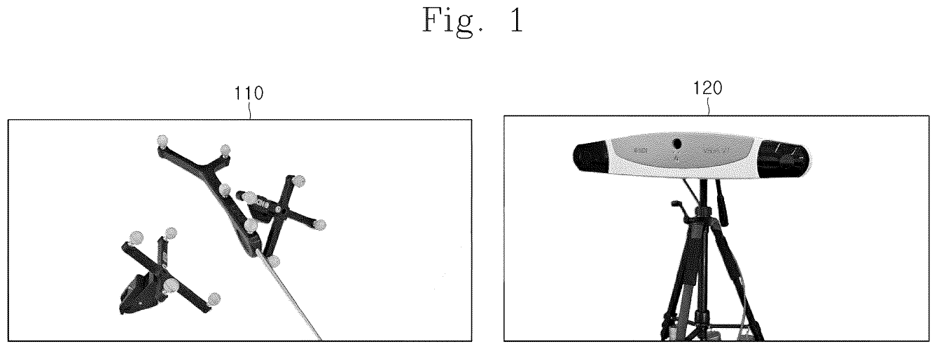

FIG. 1 illustrates an infrared (IR) marker 110 and an infrared camera 120 which are used in IR marker-based augmented reality object registration of the related art.

The registration using existing IR markers is point and surface-based registration and often causes errors in the center coordinates of an IR marker scan model due to the slice interval and imaging direction of computed tomography (CT), resulting in inaccurate registration.

When an IR marker is attached to the body of a patient and then a 3D model of the patient (or a body part of the patient), including the IR marker, is reconstructed through a CT scan, the 3D model is scanned based on the center of the IR marker and the model reconstructed in 3D is registered to a template marker model (point-based registration).

However, since the registration result is inaccurate only with point-based registration, surface-based registration is further performed.

The point cloud for the surface is collected by rubbing the probe of the IR marker over the skin. Then, additional registration is performed using Iterative Closest Point, based on the point cloud information collected for the surface (surface-based registration).

However, the hardness of the skin is generally not very high, and thus deformation caused by skin compression occurring during the process of rubbing the probe against the skin may prevent point information on the surface from being collected normally. Furthermore, when the amount of collected point data is insufficient, performing Iterative Closest Point may lead to the collected points acting as point noise, making the registration even less accurate.

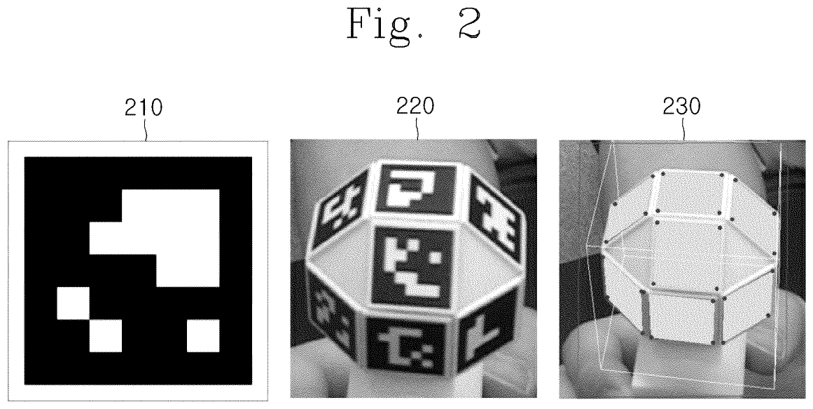

FIG. 2 shows an example of an ArUco marker used in ArUco marker-based augmented reality object registration of the related art.

An ArUco marker 210 is a type of binary square reference marker used in camera pose estimation, object tracking, etc. Each marker has a unique identification number or symbol, allowing differentiation between markers. Unlike the IR marker that tracks a marker in a shared field of view between cameras, the ArUco marker enables marker tracking even with only one camera.

However, since the ArUco marker is used after 2D printed on printable materials such as paper or Foamex (2D printing only on the surface of the material), the pattern of the marker may not be visible in the CT scan results. In other words, the ArUco marker 220 with an Arco pattern printed on the surface may not have the pattern visible in the CT scan result 230.

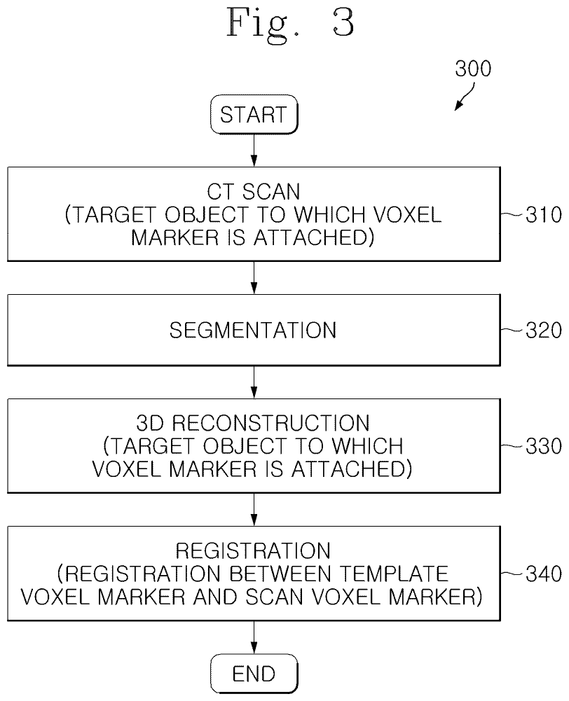

FIG. 3 is a flowchart illustrating a method of registering an augmented reality object, based on a voxel marker according to an embodiment of the present disclosure.

A voxel marker-based augmented reality object registration method 300 according to one embodiment of the present disclosure includes performing a CT scan of a target object (mainly, a patient or an affected area of a patient) to which a voxel marker is attached (310), performing segmentation by which only a region of interest of the target object and a voxel marker are extracted from the CT scan image (320), reconstructing the segmented CT scan image (2D image) into a 3D model (330), and performing registration by aligning the voxel marker (scan voxel marker) generated by the CT scan with the location, size, and rotation of template voxel marker (340). Here, the template voxel marker is a 3D voxel marker generated to correspond to an actual manufactured voxel marker and is an ideal 3D model voxel marker that reflects the location, size, and rotation of an actual voxel marker.

In performing of the CT scan of the target object to which the voxel marker is attached (310), a voxel marker is attached to at least one location on the target object in a state in which the target object, which corresponds to a patient, is fixed on the CT scanner table to be immobile. In this state, the CT scanner rotates circularly while performing X-ray imaging (CT scanning) of the target object. As a result of the CT scan, multiple 2D CT scan images of the target object having the voxel marker attached thereto are generated. The term “voxel” is a combination of “volume” and “pixel” and refers to the minimum unit in a 3D space used in computer science. A voxel is expressed in the shape of a cube. However, the voxel does not refer to a physical object and is a unit for handling 3D data. Here, a physical voxel marker to be attached to a target object may not necessarily be divided into multiple cube-shaped voxels. However, since a reconstructed 3D voxel marker model obtained through the CT scan is composed of voxel units, in the description of the embodiments of the present disclosure, the physical marker to be attached to a target object is referred to as a voxel marker.

In performing the segmentation by which only a region of interest of the target object and a voxel marker are extracted from the CT scan image (320), only the region of interest of the target object and the voxel marker are extracted from each 2D CT scan image, and the remaining parts are removed. In this regard, the region of interest of the target object corresponds to a region that is worth expressing on augmented reality content, such as a patient's affected area or a surgical site. As a result, a 2D CT scan image in which the region of interest of the target object and the voxel marker are extracted is generated.

In reconstructing the segmented CT scan image (2D image) into a 3D model (330), a 3D model is produced by integrating multiple 2D CT scan images from which unnecessary or irrelevant regions have been removed through segmentation. That is, a set of 2D scan voxel markers obtained from the multiple 2D CT scan image sets that have undergone segmentation is stacked in a 3D space and reconstructed as a 3D scan voxel marker, and a set of 2D target objects obtained from the multiple 2D CT scan image sets that have undergone segmentation is stacked in a 3D space and reconstructed as a 3D scan target object. The 3D scan target object may be reconstructed as a polygonal 3D model through an algorithm such as Marching Cube (it is not limited to the polygonal 3D model and may also be reconstructed as a 3D voxel model). Since generating a 3D image by combining multiple 2D CT scan images corresponds to related art, a detailed description thereof will be omitted.

In performing of registration by aligning the voxel marker (scan voxel marker) generated by the CT scan to the location, size, and rotation of the template voxel marker (340), the registration between the scan voxel marker and the template voxel marker may be achieved by two registrations. The primary registration is coarse registration in which a rigid transformation matrix is applied to the scan voxel marker such that the scan voxel marker is initially registered to the template voxel marker, thereby ensuring that the location and rotation of the scan voxel marker are as similar as possible to those of the template voxel marker. However, since the accuracy of the registration only through the primary registration is insufficient, second registration is further performed. The secondary registration is additional registration performed to minimize the registration error between the scan voxel marker and the template voxel marker after the primary registration. In the secondary registration, registration is performed so that the brightness difference between each voxel included in the scan voxel marker and each voxel included in the template voxel marker is minimized. The brightness difference between voxels refers to the luminance difference between voxels, and the brightness difference between voxels plays a role in representing the shape or location of the target object. That is, since the brightness value of a voxel represents the density difference of the target object (for example, a high brightness value may represent a high-density tissue such as bone, and a low brightness value may represent a low-density tissue such as soft tissue), the brightness value of a voxel may be used to identify the relative location of the voxel within the target object. Ultimately, by adjusting (registering) the location and angle of the scan voxel marker to minimize the difference in brightness values of a large number of voxels contained in each of the scan voxel marker and the template voxel marker, the coordinate system difference between the 3D CT scan image and the template voxel marker is minimized, thereby ensuring that the location, size, and rotation of the 3D CT scan image closely match those of the actual object as much as possible.

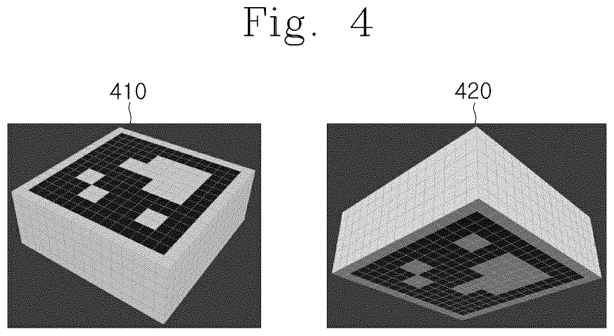

FIG. 4 illustrates a template voxel marker manufactured according to an embodiment of the present disclosure.

The voxel marker of the present disclosure is configured such that the upper surface 410 and the lower surface 420 include the same pattern, and even if the middle part of the marker is cut, the pattern appearing on the cut surface remains identical to the pattern on the upper or lower surface. That is, the voxel marker of the present disclosure is manufactured so that the ArUco pattern defines a columnar shape, and the ArUco pattern region is made of a material having a different density from that of the area excluding the corresponding pattern portion. In addition, the voxel marker of the present disclosure may be divided into countless voxels, and the number of voxels is determined according to the resolution.

When generating a 3D marker, based on a point cloud, computations need to be performed for each point, resulting in a high computational load. Since points in the point cloud have no volume and exist as a single point, infinitely magnifying the model reveals gaps between the points, thus making it difficult to accurately represent the model.

On the other hand, a voxel-based 3D marker may be considered that a certain set of point clouds from a point cloud-based model is enclosed in a single voxel, and a brightness value is assigned to that voxel (or alternatively, each 3D voxel may be assigned an individual brightness value). Since computations are performed per voxel rather than per point, the computational load is reduced. In addition, the voxel has a hexahedral shape, which is similar to the shape of a marker due to the structural characteristics thereof, the accuracy of registration based on the marker is also higher. For example, when a voxel-based 3D marker consists of two types of voxels, which is a white voxel and a black voxel, the white voxel may be assigned a brightness value of 1, while the black voxel may be assigned a brightness value of 0.

Based on that high-density objects appear white and low-density objects appear black in CT scans, the white part of the voxel-based 3D marker may be made with high-density material, and the black part may be made with low-density material (multiple densities can also be distinguished by using two or more different materials). As a result, the voxel-based 3D marker that reflects density differences maintains marker pattern thereof even in CT scans.

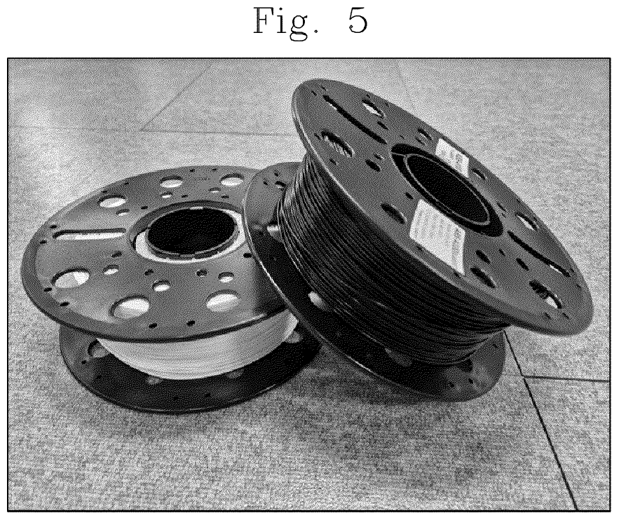

FIG. 5 illustrates a 3D printing filament material used in manufacturing a voxel-based marker having a density difference obtained through a combination of materials, according to an embodiment of the present disclosure.

In the example of FIG. 5, a high-density object may be manufactured using a white filament and a low-density object may be manufactured using a black filament.

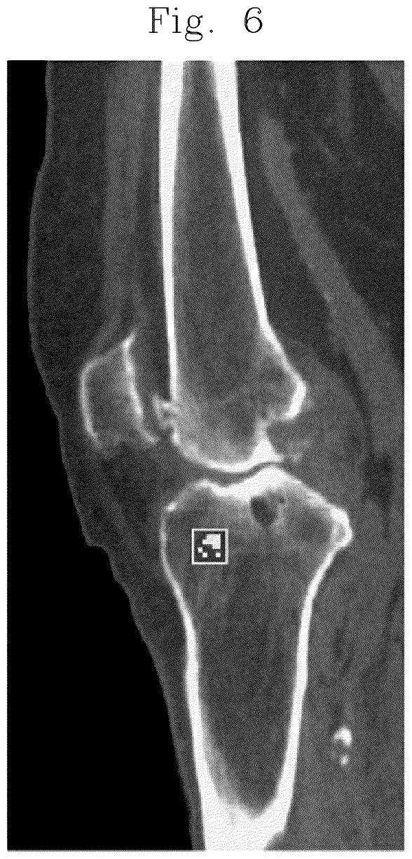

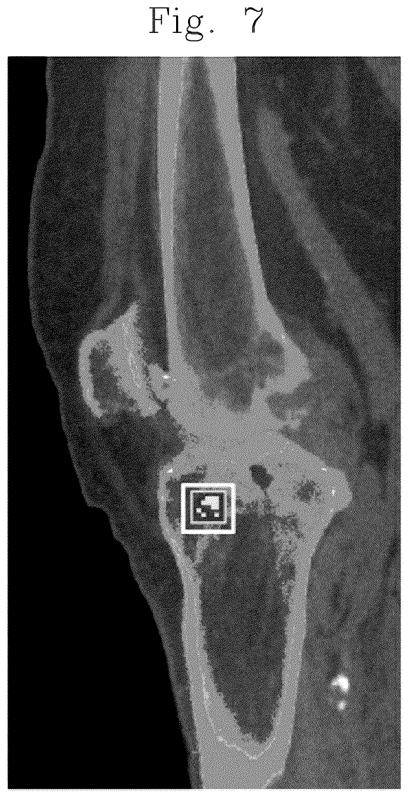

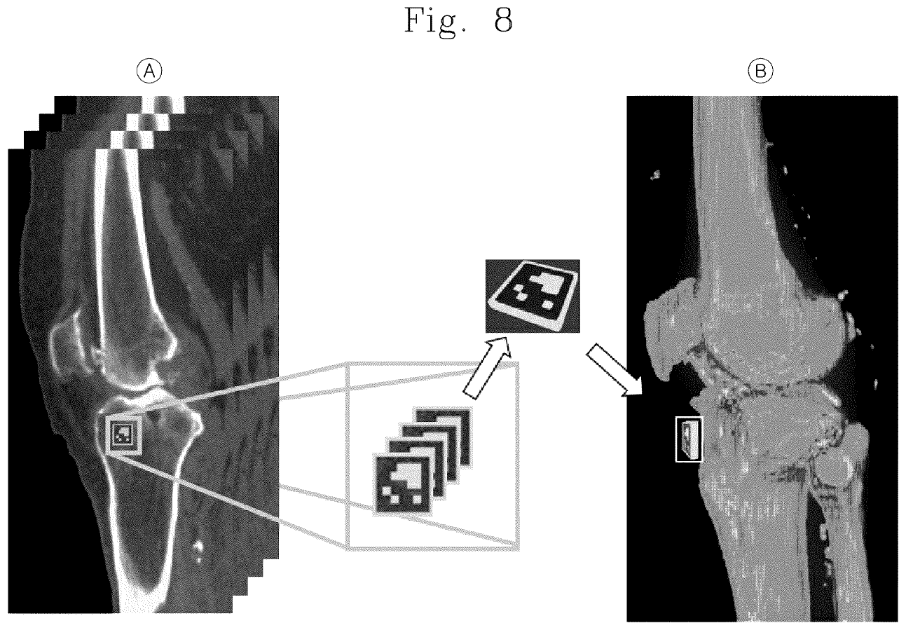

FIG. 6 illustrates a 2D scan image obtained by CT scanning a target object to which a voxel-based 3D marker is attached, according to an embodiment of the present disclosure, FIG. 7 illustrates an image that has undergone segmentation by which only a region of interest of a target object and a voxel marker are extracted from a CT scan image, according to an embodiment of the present disclosure, and FIG. 8 illustrates a 3D model generated by integrating a plurality of 2D CT scan images that have undergone segmentation, according to an embodiment of the present disclosure.

FIG. 8\textcircled{a} shows multiple 2D CT scan image sets (e.g., DICOM image sets) that have undergone segmentation, and FIG. 8\textcircled{b} shows that a set of 2D scan voxel markers obtained from the multiple 2D CT scan image sets that have undergone segmentation is stacked in a 3D space and reconstructed as a 3D scan voxel marker, and a set of 2D target objects obtained from the multiple 2D CT scan image sets that have undergone segmentation is stacked in a 3D space and reconstructed as a 3D scan target object. The 3D scan target object may be reconstructed as a polygonal 3D model through an algorithm such as Marching Cube (it is not limited to the polygonal 3D model and may also be reconstructed as a 3D voxel model). Since generating a 3D image by combining multiple 2D CT scan images corresponds to related art, a detailed description thereof will be omitted.

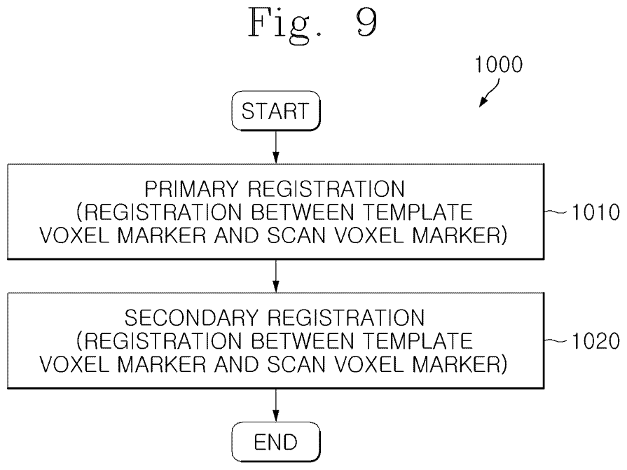

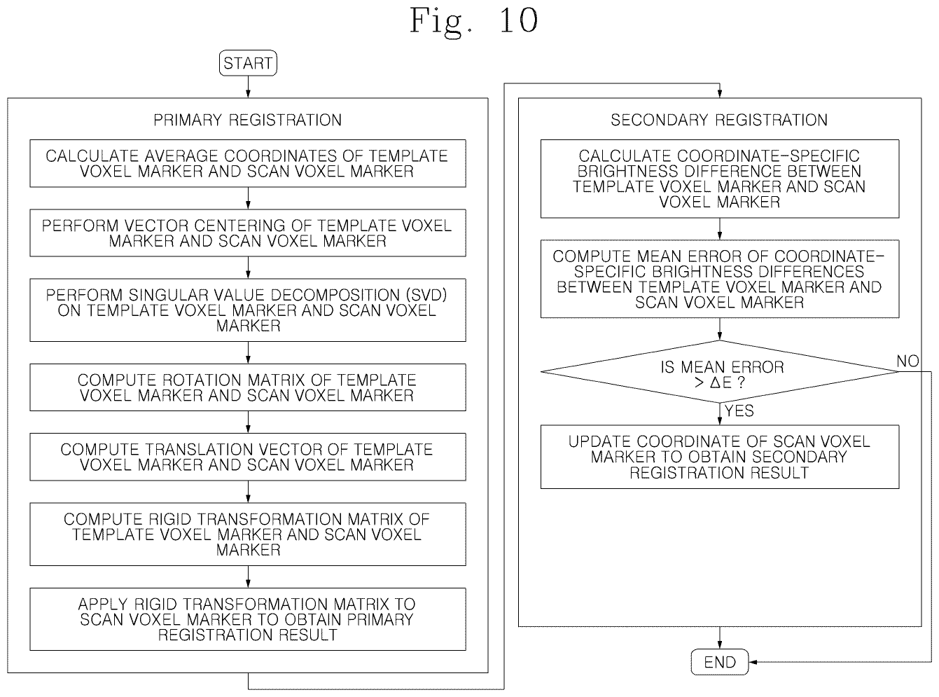

FIG. 9 is a flowchart (including primary and secondary registration) for aligning and registering voxel markers generated by a CT scan with template voxel markers according to an embodiment of the present disclosure, and FIG. 10 is a flowchart illustrating operations constituting primary registration and secondary registration by which a voxel marker generated by a CT scan is aligned and registered with a template voxel marker according to an embodiment of the present disclosure.

The scan voxel marker may be registered to the template voxel marker in two stages. The primary registration 1010 is coarse registration in which a rigid transformation matrix is applied to the scan voxel marker such that the scan voxel marker is initially registered to the template voxel marker (the location and rotation of the scan voxel marker are as similar as possible to those of the template voxel marker).

However, since the accuracy of the registration only through the primary registration is insufficient, secondary registration is further performed. The secondary registration 1020 is additional registration performed to minimize the registration error between the scan voxel marker and the template voxel marker after the primary registration.

In the secondary registration, registration is performed so that the brightness difference between each voxel included in the scan voxel marker and each voxel included in the template voxel marker is minimized. The brightness difference between voxels refers to the luminance difference between voxels, and the brightness difference between voxels plays a role in representing the shape or location of the target object. That is, since the brightness value of a voxel represents the density difference of the target object (for example, a high brightness value may represent a high-density tissue such as bone, and a low brightness value may represent a low-density tissue such as soft tissue), the brightness value of a voxel may be used to identify the relative location of the voxel within the target object. Ultimately, by comparing the difference in brightness values of a large number of voxels included in each of the scan voxel markers and the template voxel markers and adjusting (registering) the location and angle of the scan voxel marker to the location and angle at which the difference is minimized, the coordinate system difference between the scan voxel marker and the template voxel marker is minimized, thereby minimizing the coordinate system difference between the 3D CT scan image and the actual object (the actual object based on the template voxel marker) and ensuring that the location and rotation of the 3D CT scan image closely match those of the actual object as much as possible.

The primary and secondary registration are described in more detail with reference to FIG. 10.

In the primary registration, the average coordinates of the template voxel marker and the scan voxel marker are first calculated.

$$\overline{S} = \frac{1}{n} {\sum }_{i = 1}^{n} {S}_{i}$$

$$\overline{T} = \frac{1}{n} {\sum }_{i = 1}^{n} {T}_{i}$$

Here, Si (i=1, 2, . . . , n) is the scan voxel coordinate, Ti (i=1, 2, . . . , n) is the template voxel coordinate, n is the total number of voxels, S̅ is the scan model average coordinate, and T̅ is the template marker model average coordinate. At this time, Si and Ti may be expressed as follows.

$${S}_{i} = [ \begin{matrix}{x}_{i} \\ {y}_{i} \\ {z}_{i}\end{matrix} ]$$

$${T}_{i} = [ \begin{matrix}{x}_{i}^{′} \\ {y}_{i}^{′} \\ {z}_{i}^{′}\end{matrix} ]$$

Then, vector centering is performed on the template voxel marker and the scan voxel marker.

$$\tilde{S} = {S}_{i} - \overline{S}$$

$$\tilde{T} = {T}_{i} - \overline{T}$$

Here, \tilde{S}ᵢ (i=1, 2, . . . , n) is the centered vector of the scan voxel marker, and \tilde{T}ᵢ (i=1, 2, . . . , n) is the centered vector of the template voxel marker.

Then, singular value decomposition (SVD) is performed on the template voxel marker and the scan voxel marker.

$$H = U \sum {V}^{T}$$

Here, H is the covariance matrix and is calculated as follows.

$$H = {{\sum }^{n}}_{i = 1} {\tilde{S}}_{i} {\tilde{T}}_{1}^{T}$$

Here, UΣV^{T} is the result of applying SVD to the covariance matrix.

In this case, U, Σ, and V may be represented as follows.

$$U = [ \begin{matrix}{u}_{11} & {u}_{12} & {u}_{13} \\ {u}_{21} & {u}_{22} & {u}_{23} \\ {u}_{31} & {u}_{32} & {u}_{33}\end{matrix} ]$$

$$\sum = [ \begin{matrix}{σ}_{1} & 0 & 0 \\ 0 & {σ}_{2} & 0 \\ 0 & 0 & {σ}_{3}\end{matrix} ]$$

$$V = [ \begin{matrix}{v}_{11} & {v}_{12} & {v}_{13} \\ {v}_{21} & {v}_{22} & {v}_{23} \\ {v}_{31} & {v}_{32} & {v}_{33}\end{matrix} ]$$

Next, the rotation matrix of the template voxel marker and the scan voxel marker is calculated.

R=VU^{T} (When det(R)<0, the sign of the third column of V is flipped and recalculated.)

Here, R is the rotation matrix and may be represented as follows.

$$R = [ \begin{matrix}{r}_{11} & {r}_{12} & {r}_{13} \\ {r}_{21} & {r}_{22} & {r}_{23} \\ {r}_{31} & {r}_{32} & {r}_{33}\end{matrix} ]$$

Next, the translation vector of the template voxel marker and the scan voxel marker is calculated.

$$t = \overline{T} - R \overline{S}$$

Here, t is the translation vector.

Next, the rigid transformation matrix of the template voxel marker and the scan voxel marker is calculated.

$$X = [ R \vert t ]$$

Here, X is the rigid transformation matrix.

Next, the rigid transformation matrix is applied to the scan voxel marker to obtain a primary registration result.

$${S}_{new} = {XS}_{old}$$

Here, S_{old} is the scan voxel marker matrix before the primary registration, and S_{new} is the scan voxel marker matrix after the primary registration.

In the following secondary registration, the coordinate-specific brightness difference between the template voxel marker and the scan voxel marker is first calculated.

$${E}_{a} ( p ) = \frac{1}{n} {\sum }_{i = 1}^{n} {( {I}_{c} ( {S}_{i} ) - {I}_{c} ( {T}_{i} ) )}^{2}$$

Here, Eₐ(p) is an error function representing the brightness difference between models at arbitrary coordinates p, and I_{c}(p) is the brightness at arbitrary coordinates p, which has a constant value because it is a grayscale CT image.

Next, the mean square error of the coordinate-specific brightness of the template voxel marker and the scan voxel marker is calculated.

$${arg min}_{Δ p} \frac{1}{n} {\sum }_{i = 1}^{n} {( {I}_{c} ( {S}_{i} + Δ p ) - {I}_{c} ( {T}_{i} ) )}^{2}$$

Here, Δₚ is the coordinate change.

When the mean square error calculated by the formula above is greater than a predetermined reference value (ΔE), the coordinates of the scan voxel marker are updated to produce the secondary registration result. At this time, the predetermined reference value (ΔE) is a value that may be configured according to the precision or resolution requirements (when high precision is required, the reference value may be lowered, and when low precision is tolerated, the reference value may be higher).

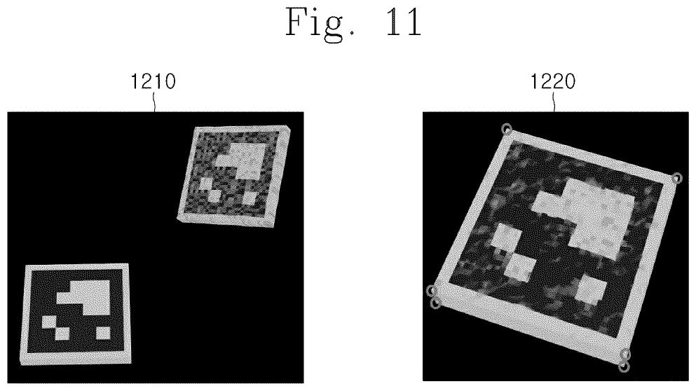

FIG. 11 illustrates a process of primary registration for aligning a voxel marker generated by a CT scan with a template voxel marker according to an embodiment of the present disclosure.

In given reference numeral 1210, the lower left is a template voxel marker, and the upper right is a scan voxel marker. After going through the primary registration described above, the eight corners of the scan voxel marker are registered, producing a primary registration result as shown in given reference numeral 1220.

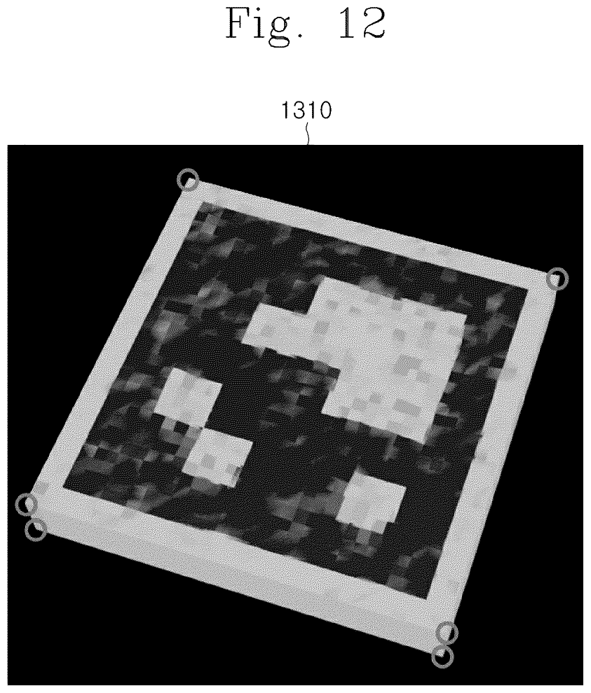

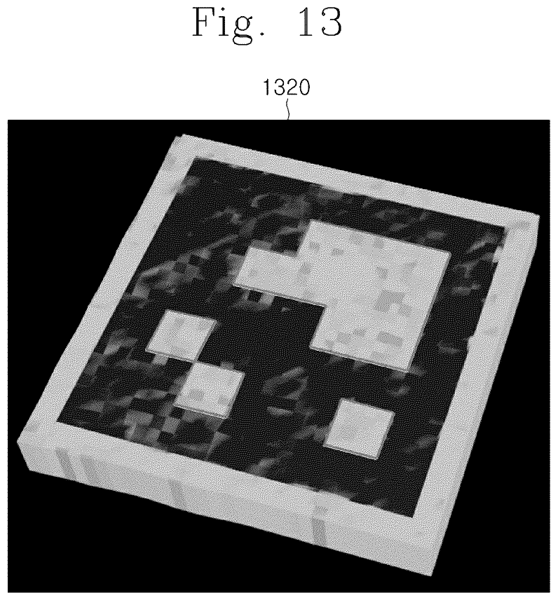

FIG. 12 illustrates a primary registration result obtained by aligning a voxel marker generated by a CT scan with a template voxel marker according to an embodiment of the present disclosure, and FIG. 13 illustrates a secondary registration result obtained by aligning a voxel marker generated by CT scans with a template voxel marker according to an embodiment of the present disclosure.

As noted from the primary registration result shown in FIG. 12, voxels defining the scan voxel marker are partially displayed as hexahedral shape by scanning (blurred). In addition, the display color is displayed as a white part (high-density part) and a black part (low-density part) due to the density difference of the voxels defining the scan voxel marker. However, it is difficult to display the shape of each voxel or the boundary between white and black to the level of the template voxel marker 1210 at the lower left in FIG. 11 due to the performance of the CT apparatus.

When the secondary registration is performed based on the primary registration result shown in FIG. 12, the location, size, and rotation (angle) of the scan voxel marker are adjusted to be closer to those of the template voxel marker, and the final registration result is produced as shown in FIG. 13. As illustrated in FIG. 13, secondary registration result is achieved so that, compared to the primary registration (only the 8 corners of the scan voxel marker are registered to the template), the scan voxel marker that has undergone the secondary registration has the ArUco patterns at the top and bottom and the location, size, and rotation (angle) of the cross-sectional pattern inside the scan voxel marker adjusted to be closer to the template voxel marker.

Apparatus to which the Proposed Method of the Present Disclosure is Applicable

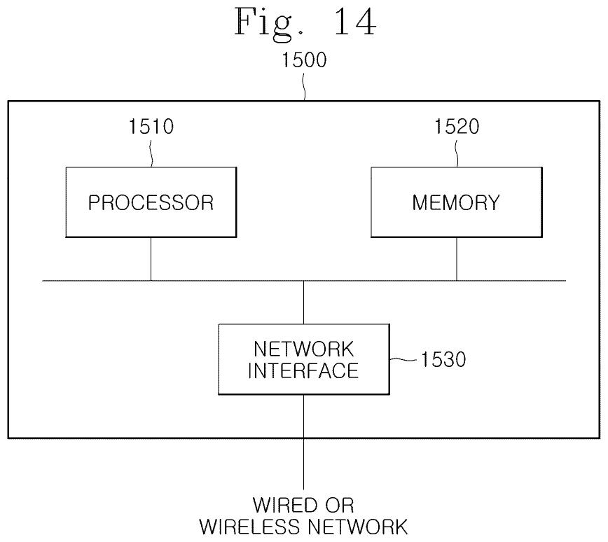

FIG. 14 illustrates an apparatus 1500 to which the proposed method of the present disclosure is applicable. The apparatus 1500 may correspond to a server or terminal which is configured to align an augmented reality object, based on a voxel marker.

Referring to FIG. 14, the apparatus 1500 may be a server apparatus or terminal apparatus configured to implement a process for a method of registering an augmented reality object, based on a voxel marker.

For example, the apparatus 1500 to which the proposed method of the present disclosure is applicable may include a network apparatus, such as a repeater, a hub, a bridge, a switch, a router, and a gateway, a computer apparatus, such as a desktop computer and a workstation, a mobile terminal such as smartphone, a portable apparatus such as a laptop computer, a home appliance such as a digital TV, and a transportation means such as an automobile. As another example, an apparatus 1200 to which the present disclosure is applicable may be included as a part of an application specific integrated circuit (ASIC) implemented in the form of a system on chip (SOC).

A memory 1520 may be operatively connected to a processor 1510 and store programs and/or instructions for processing and controlling the processor 1510. The memory may store data and information used in the present disclosure, control information required for data and information processing according to the present disclosure, temporary data generated during data and information processing, etc. The memory 1520 may be implemented as a storage apparatus such as a ROM (Read Only Memory), a RAM (Random Access Memory), an EPROM (Erasable Programmable Read Only Memory), an EEPROM (Electrically Erasable Programmable Read-Only Memory), a flash memory, a SRAM (Static RAM), an HDD (Hard Disk Drive), an SSD (Solid State Drive), etc.

The processor 1510 may be operatively connected to the memory 1520 and a network interface 1530, and controls the operation of each module within the apparatus 1500. In particular, the processor 1510 may perform various control functions for performing the proposed method of the present disclosure. The processor 1510 may also be called a controller, a microcontroller, a microprocessor, a microcomputer, etc. The proposed method of the present disclosure may be implemented by hardware, firmware, software, or a combination thereof. When the present disclosure is implemented using hardware, an ASIC (application specific integrated circuit) or a DSP (digital signal processor), a DSPD (digital signal processing apparatus), a PLD (programmable logic apparatus), an FPGA (field programmable gate array), etc. configured to perform the present disclosure may be equipped in the processor 1510. When implementing the proposed method of the present disclosure using firmware or software, the firmware or software may include instructions related to modules, procedures or functions that perform functions or operations necessary for implementing the proposed method of the present disclosure, and the instructions may be stored in the memory 1520 or stored in a computer-readable recording medium (not shown) separate from the memory 1520 and, when executed by the processor 1510, the apparatus 1500 may be configured to implement the proposed method of the present disclosure.

In addition, the apparatus 1500 may include a network interface apparatus 1530. During operation, the network interface apparatus 1530 may be connected to the processor 1510, and the processor 1510 may control the network interface apparatus 1530 to transmit or receive wireless/wired signals carrying information and/or data, signals, messages, etc. via a wireless/wired network. The network interface apparatus 1530 may support various communication standards, such as IEEE 802 series, 3GPP LTE(-A), 3GPP 5G, etc. and transmit and receive control information and/or data signals according to the communication standards. The network interface apparatus 1530 may also be implemented outside the apparatus 1500 as needed.

The embodiments and drawings described in this specification are merely exemplary and do not limit the scope of the present disclosure in any way. In addition, the lines or connection members between the components illustrated in the drawings are merely exemplary of functional connections and/or physical or circuit connections, and may be replaced or represented as additional and various functional connections, physical connections, or circuit connections in an actual apparatus. In addition, unless explicitly stated as “essential” or “important,” the component may not be required for the application of the present disclosure.

The use of the term “above” and similar referential terms in the specification of the present disclosure (especially in the claims) may correspond to both singular and plural. In addition, when a range is specified in the present disclosure, the disclosure includes embodiments that apply individual values within that range (unless stated otherwise). This is equivalent to explicitly describing each individual value constituting the range in the detailed description of the disclosure. In addition, the operations presented in method claims of the present disclosure are not necessarily intended to be restricted to a specific order. Unless the nature of a particular process inherently requires a preceding operation, the sequence of operations may be appropriately modified as needed. Furthermore, all examples or exemplary terms (e.g., “for example,” “etc.”) in the present disclosure are provided solely for the purpose of detailed explanation and do not limit the scope of the disclosure unless explicitly defined in the claims. A person skilled in the art would understand that various modifications, combinations, and alterations may be made within the scope of the claims or their equivalents, depending on design conditions and elements.

1. A method of registering an augmented reality object, based on a voxel marker, the method comprising:
performing a CT scan of a target object to which a voxel marker is attached;
performing segmentation by which only a region of interest of the target object and the voxel marker are extracted from a CT scan image;
reconstructing the segmented CT scan image into a 3D model; and
performing registration by aligning a voxel marker generated by the CT scan with a location, size, and rotation of a template voxel marker.

2. The method of claim 1, wherein the voxel marker comprises multiple types of voxels having different densities.

3. The method of claim 1, wherein the region of interest comprises an affected area or surgical target area of a patient.

4. The method of claim 1, wherein the template voxel marker is an ideal 3D model voxel marker that reflects the location, size, and rotation of the voxel marker.

5. The method of claim 1, wherein the performing of the registration comprises performing primary registration and secondary registration,
wherein the performing of the primary registration comprises registering the voxel marker generated by the CT scan with the template voxel marker by applying a rigid body transformation matrix thereto, and
wherein the performing of the secondary registration comprises performing registration to minimize a brightness difference between each voxel included in the voxel marker generated by the CT scan and each voxel included in the template voxel marker.

6. A computer program stored in a medium to execute, in combination with hardware, the method of claim 1 for registering an augmented reality object, based on a voxel marker.

7. An apparatus for registering an augmented reality object, based on a voxel marker, the apparatus comprising a processor,
wherein the processor is configured to execute:
a CT scan of a target object to which a voxel marker is attached;
segmentation by which only a region of interest of the target object and the voxel marker are extracted from a CT scan image;
reconstruction of the segmented CT scan image into a 3D model; and
registration by aligning the voxel marker generated by the CT scan with a location, size, and rotation of a template voxel marker.

8. The apparatus of claim 7, wherein the voxel marker comprises multiple types of voxels having different densities.

9. The apparatus of claim 7, wherein the region of interest comprises an affected area or surgical target area of a patient.

10. The apparatus of claim 7, wherein the template voxel marker is an ideal 3D model voxel marker that reflects the location, size, and rotation of the voxel marker.

11. The apparatus of claim 7, wherein the executing of the registration comprises executing primary registration and secondary registration,
wherein the executing of the primary registration comprises registering the voxel marker generated by a CT scan with the template voxel marker by applying a rigid body transformation matrix thereto, and
wherein the executing of the secondary registration comprises executing registration to minimize a brightness difference between each voxel included in the voxel marker generated by a CT scan and each voxel included in the template voxel marker.
